(19) United States
(12) Patent Application Publication
(10) Pub. No.: US 2026/0021022 A1
(43) Pub. Date: Jan. 22, 2026

(54) Zirconia Green Body with Submicron Grain Control for Rapid Sintering and High-Throughput Dental Restorations

(71) Applicant: B & D Dental Corporation, West Valley City, UT (US)

(72) Inventors: Benjamin JUNG, West Valley City, UT (US); Daniel Yonil JUNG, West Valley City, UT (US)

(21) Appl. No.: 19/274,724

(22) Filed: Jul. 21, 2025

Related U.S. Application Data
(60) Provisional application No. 63/674,157, filed on Jul. 22, 2024.

(51) Int. Cl. A61K 6/818; C04B 35/626; C04B 35/64

(52) U.S. Cl. CPC A61K 6/818; C04B 35/6261; C04B 35/64; C04B 2235/3244; C04B 2235/785

(57) ABSTRACT

A dental zirconia-based green body comprises a zirconium oxide and at least one other non-zirconium oxide. The at least one other non-zirconium oxide equaling 7-20 weight percent (wt %) based on a total weight percent of the zirconia-based green body. The zirconia-based green body being sinterable to form a sintered body with sintered grains. All the sintered grains of the sintered body have a sintered grain size less than 1000 nano-meters (nm). A majority of the sintered grains of the sintered body have a sintered grain size greater than 100 nm.

### PRIORITY CLAIM(S)

Priority is claimed to U.S. Provisional Patent Application Ser. No. 63/674,157, filed Jul. 22, 2024, which is incorporated herein by reference.

### BACKGROUND OF THE INVENTION

### 1. Field of the Invention

The present invention relates to zirconia-based green bodies for dental use.

### 2. Description of the Related Art

Currently, most dental zirconia green bodies are sintered between 1500-1600° C., resulting in sintered grain sizes of 500-2000 nm, causing several problems. High sintering temperature not only takes more time but also leads to larger grain sizes, thus weakening the flexural strength and causing abrasiveness to the opposing tooth, which is not beneficial for patients.

### SUMMARY OF THE INVENTION

The present technology relates to zirconia-based green bodies for dental use, particularly those suitable for fast sintering under standard atmospheric conditions. In addition, the present technology relates to zirconia compositions capable of producing multiple dental restorations—such as crowns, bridges, or All-on-X prostheses—within one hour of sintering. The present technology can ensure that no sintered grain exceeds 1,000 nanometers (nm), thereby improving mechanical strength, translucency, and sintering uniformity. The present technology can further enable low-temperature sintering at 1,300-1,400 degrees Celsius (° C.) while maintaining flexural strength above 800 megapascals (MPa).

Currently, most dental zirconia green bodies are sintered between 1500-1600° C., resulting in sintered grain sizes of 500-2000 nm, causing several problems. High sintering temperature not only takes more time but also leads to larger grain sizes, thus weakening the flexural strength and causing abrasiveness to the opposing tooth, which is not beneficial for patients. The present technology can: 1) reduce the particle size of the green body ultimately reducing the grain size of the sintered body; 2) significantly increase the number of restorations that can be sintered at once; and 3) lower the sintering temperature and time, and reduce abrasiveness to the patient's opposing teeth.

U.S. Pat. No. 9,737,383 (Tosoh) discloses a translucent zirconia sintered body, with 4.0 mol % (=7.1 wt %) to 6.5 mol % of yttria (=11.3 wt %), in which the average grain size is preferably in the range of 0.3 to 1.0 μm, and more preferably between 0.4 and 0.8 μm. No maximum grain size is disclosed. Publicly available information published by Tosoh appear to show microstructures in which a plurality of the grains exceeds 1.0 μm in diameter, based on SEM imagery.

In contrast, the present technology explicitly limits the maximum grain size of the sintered zirconia body to less than 1,000 nm (1.0 μm), as confirmed by scanning electron microscope (SEM) analysis. The SEM images of the present technology demonstrate that none of the grains exceed 1.0 μm. The present technology provides a finely controlled grain structure with distinct technical advantages in translucency, mechanical strength, and compatibility with high-throughput multi-unit sintering.

Conventional 3 mol % yttria-stabilized zirconia (3YSZ), containing no more than 4.0 mol % yttria (approximately 7 wt %), is known to exhibit sintered grain sizes consistently below 1,000 nanometers. However, due to its relatively low yttria content, 3YSZ appears overly white and highly opaque, and thus lacks translucency for esthetic dental applications. As a result, since the introduction of Tosoh's 4YS zirconia in 2014, the relevance of 3YSZ in the dental field has sharply declined.

Moreover, 3YSZ typically requires high-temperature sintering at or above 1,500° C. for at least 7 hours, making it highly inefficient and unsuitable for multi-case sintering workflows for fast sintering. In contrast, the present technology enables controlled translucency through a refined microstructure while ensuring that no sintered grain exceeds 1,000 nanometers, even in zirconia compositions containing higher yttria content (e.g., 4 mol % or more). Therefore, both in terms of material composition and sintering behavior, the present technology is fundamentally distinct from conventional 3YS zirconia.

While many zirconia manufacturers have introduced products marketed as “fast sintering,” such claims are often made with significant limitations. One common restriction is that such fast sintering is only applicable to crowns or bridges with wall thicknesses of 3 mm or less. This is primarily due to the fact that these materials often contain a substantial portion of sintered grains exceeding 1,000 nanometers, and their grains can be irregularly shaped and unevenly distributed. Such microstructural inconsistency can lead to residual pores trapped between grains during fast sintering, which in turn can result in increased opaqueness and reduced translucency.

Another unspoken but critical limitation in current fast-sintering products is that manufacturers typically do not disclose the maximum number of units that can be sintered simultaneously within a given time frame. This omission stems from the fact that large and disordered grain structures can be prone to trapping air during rapid heating, which can contribute to undesirable brightness and optical opaqueness in the final restoration. While some products may successfully sinter one or a few restorations, they do not disclose or support consistent high-throughput performance. In contrast, the present technology provides a clear numerical performance benchmark—such as the ability to sinter 20, 30, or more units in one hour.

The present technology provides a zirconia green body composition capable of producing multiple units of dental restorations—including crowns, bridges, and full-arch prostheses—within one hour or two hours of sintering under standard atmospheric conditions. The zirconia green body of the present technology can be characterized by a sintered microstructure in which all grain sizes remain below 1,000 nanometers and the majority fall within the range of 200 to 700 nanometers, resulting in highly uniform heat distribution and rapid sintering. This structural advantage allows the green body to be sintered in reduced time—sometimes within 30 minutes—while maintaining clinically acceptable levels of flexural strength (≥800 MPa) and optical translucency (L value ≥70). The present technology can enable the simultaneous sintering of at least 5, 10, 15, 20, 25, or even 50 dental units in a single batch; dramatically improving productivity and reducing processing time. These outcomes can be achieved without requiring special furnaces or gas atmospheres, making the invention compatible with existing dental laboratory equipment.

### BRIEF DESCRIPTION OF THE DRAWINGS

With the above and other related objectives in view, the invention consists in the details of construction and combination of parts, as will be more fully understood from the following description, when read in conjunction with the accompanying drawings in which:

FIG. 1a is a Scanning Electron Microscope (SEM) picture 100 of an example sample of a sintered body formed from a zirconia-based green body with an enlargement factor of 1.2023 that has been sintered for an hour at a highest temperature of 1350° C., according to some embodiments.

FIG. 1b is an SEM picture 150 of an example sample of a sintered body formed from a zirconia-based green body with an enlargement factor of 1.2023 that has been sintered for an hour at a highest temperature of 1375° C., according to some embodiments.

FIG. 2a is an SEM picture 200 of an example sample of a sintered body formed from a zirconia-based green body with enlargement factor of 1.2023 that has been sintered for 5 hours at a highest temperature of 1400° C., according to some embodiments.

FIG. 2b is an SEM picture 250 of an example sample of a sintered body formed from a zirconia-based green body with enlargement factor of 1.2023 that has been sintered for an hour at a highest temperature of 1400° C., according to some embodiments.

FIG. 3 is an SEM picture 300 of an example sample of a sintered body formed from a zirconia-based green body with enlargement factor of 1.2015 that has been sintered for 3 hours at a highest temperature at 1400° C., according to some embodiments.

FIG. 4 is an SEM picture 400 of an example sample of a sintered body formed from a zirconia-based green body with enlargement factor of 1.2015 that has been sintered for 7 hours at a highest temperature at 1400° C., according to some embodiments.

FIG. 5 is an SEM picture 500 of a prior art sample of a sintered body formed from a zirconia-based green body with enlargement factor of 1.2230 that has been sintered for 1.5 hours at a highest temperature at 1560° C.

FIG. 6 is an SEM picture 600 of a prior art sample of a sintered body formed from a zirconia-based green body with enlargement factor of 1.2319 that has been sintered for 7.5 hours at a highest temperature at 1510° C.

FIG. 7 is an SEM picture 700 of a prior art sample of a sintered body formed from a zirconia-based green body with enlargement factor of 1.2440 that has been sintered for 1 hour at a highest temperature at 1500° C.

FIG. 8 is an SEM picture 800 of a prior art sample of a sintered body formed from a zirconia-based green body with enlargement factor of 1.2450 that has been sintered for 7 hours at a highest temperature at 1500° C.

FIG. 9 is a diagram of an example sintering graph for a zirconia green body, according to some embodiments.

FIG. 10 a picture 1000 of example dental restorations that have been milled and sintered from zirconia-based green bodies, according to some embodiments. The dental restorations in columns 1 and 2 are from zirconia-based green bodies with enlargement factors of 1.2023 and 1.2015, and sintered within an hour with different sintering temperatures (as shown in Table 2), according to some embodiments. The dental restorations in columns 3 and 4 show comparative dental restorations from comparative zirconia-based green bodies with enlargement factors of 1.2450 and 1.2440, respectively.

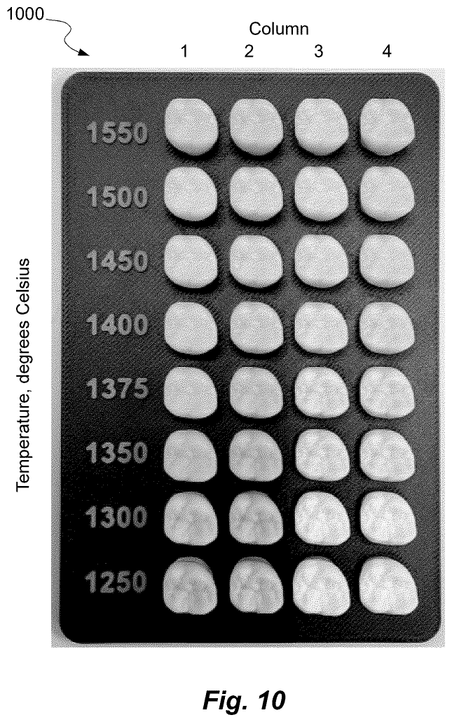

FIG. 11 is a diagram of an example graph of the incisal/occlusal strengths of dental restorations, according to the present technology.

### DETAILED DESCRIPTION OF THE EMBODIMENTS OF THE INVENTION

Illustrative embodiments of the present invention are described below. The following explanation provides specific details for a thorough understanding of and enabling description for these embodiments. One skilled in the art will understand that the invention may be practiced without such details. In some instances, well-known structures, processes, and functions have not been shown or described in detail to avoid unnecessarily obscuring the description of the embodiments.

### Definitions

It shall be noted that unless the context clearly requires otherwise, throughout the description, the words “comprise,” “comprising,” “include,” “including,” and the like are to be construed in an inclusive sense as opposed to an exclusive or exhaustive sense; that is to say, in the sense of “including, but not limited to.” Words using the singular or plural number also include the plural or singular number, respectively while adhering to the concepts of the present invention. Furthermore, references to “one embodiment” and “an embodiment” are not intended to be interpreted as excluding the existence of additional embodiments that also incorporate the recited features.

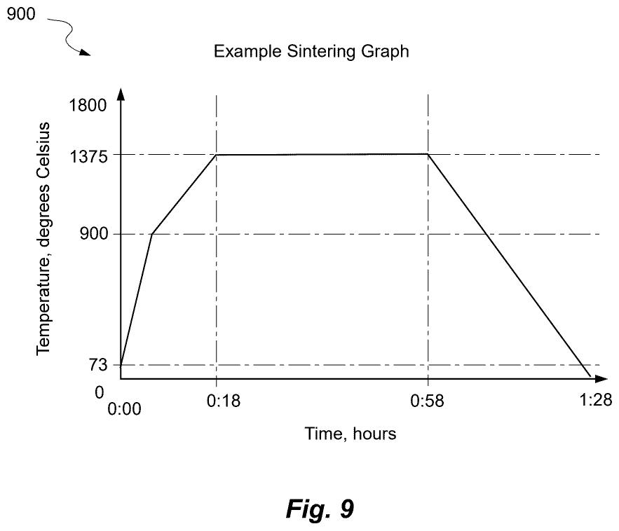

Sintering time: Sintering time within an hour means the time from the start until the holding time at the highest temperature ends (FIG. 9). In the dental industry, cooling time is generally not included in the sintering time calculation. Almost all companies calculate sintering time only until the end of the holding time. This is because, at the end of the holding time, the actual sintering itself is completed.

Total sintering time: This refers to the total time from the start of sintering to the completion of cooling. Typically, it means the time taken until the sintering graph ends at approximately 200° C.

Particle size: the diameter or characteristic dimension of an individual powder particle in its pre-sintered state. This includes both primary particles and, in some contexts, agglomerates formed during powder synthesis or handling. Particle size is typically measured in the context of raw zirconia powders, slurries, or green bodies, and is a critical factor influencing packing density, shrinkage, and sintering behavior. It is commonly characterized using techniques such as laser diffraction, dynamic light scattering, or SEM image analysis.

Sintered grain size or grain size: the diameter of crystallites, or individual grains, within a fully sintered ceramic body. After sintering, particles fuse and undergo grain growth, forming a polycrystalline solid with well-defined grain boundaries. Grain size directly affects key mechanical and optical properties such as flexural strength, translucency, and fracture toughness. It is typically measured by SEM imaging of thermally etched cross-sections or via the linear intercept method. There are various methods to measure the size of the sintered grain, but this patent application uses a simple and intuitive method. The size of a particular sintered grain size is defined as the longest distance between any two points on the boundary or perimeter of a specific grain.

Pre-sintering (also called bisque sintering or partial sintering) is a low-temperature thermal process (typically 900-1100° C.) that gives the zirconia blank enough mechanical strength for machining (e.g., milling) while keeping it soft enough to avoid tool wear. At this stage, the zirconia is porous and not fully densified.

Final sintering is the high-temperature process (typically 1350-1600° C.) that fully densifies the zirconia, eliminating porosity and forming a strong, hard, and translucent ceramic suitable for permanent dental restorations.

Standard sintering or sintering: refers to the sintering process carried out in ambient air without the use of specialized equipment or atmospheric control, such as post Hot Isostatic Pressing (HIP) processing or sintering in inert gases like argon. The sintering described in the present technology is based on standard sintering conditions typically used in conventional dental laboratories, where cost-effective and widely accessible furnaces are employed. In contrast, advanced sintering methods involving pressurized environments or inert gases may accelerate densification but are not commonly adopted in routine dental lab practice due to their high equipment cost and complexity.

Green body: As used herein, the term “green body” refers to a shaped zirconia compact that has been formed, typically by pressing or casting, but has not yet undergone final densification through high-temperature sintering. The green body possesses sufficient mechanical strength for handling and machining and generally results from a pre-sintering or partial drying process.

Crown & bridge: As used herein, the term “crown” refers to a dental restoration that covers or replaces the visible portion of a single tooth. It may be used for restorative, prosthetic, or cosmetic purposes. Unless otherwise specified, the term “crown” also encompasses bridges. The term “bridge” refers to a fixed dental prosthesis that replaces one or more missing teeth by anchoring to adjacent natural teeth or implants. A bridge typically consists of two or more crown units, including pontics and abutments, joined together as a single restoration.

Unit of restoration: As used herein, the term “unit” refers to a single tooth restoration, such as one crown, while the term “case” refers to a group of restorations processed together, which may include multiple units; for example, a 3-unit bridge is considered one case consisting of three units, and a 6-unit anterior bridge includes six units, including pontics.

All-on-X: As used herein, the term “All-on-X” (including “All-on-4”) refers to a full-arch, implant-supported dental restoration in which all teeth in either the maxillary or mandibular arch are replaced with a fixed prosthesis supported by X number of implants, typically 4 to 6. Unlike conventional bridges that replace a limited number of adjacent teeth, an All-on-X restoration functions as a single prosthetic case composed of 8 to 14 units, depending on the arch and design, with each unit corresponding to one tooth in the restored arch.

The term “Zirconia” refers to various stoichiometries for zirconium oxides, most typically ZrO2, and may also be known as zirconium oxide or zirconium dioxide. The zirconia may contain 7 wt % up to 20 weight percent of oxides of other chemical elements such as oxides of yttrium (e.g., Y2O3). Yttria (Y2O3) is an example of additives/components that stabilize zirconium oxide and increase the sintering speed or translucency of the dental blanks. Other examples include Yb2O3, CeO2, Er2O3, TiO2, B2O3, MgO, HfO2, etc.

### Description

The process steps of producing a zirconia-based green body can include:
Ball Milling: Reduce the particle size of zirconia powder using ball milling with a dispersant, running the ball mill for about 5 hours to achieve a particle size of 200-300 nm.
Making Slurry: The ball milling process creates a slurry. The term “slurry” refers to a homogeneous mixture of finely ground zirconia powder suspended in a liquid, typically water or an aqueous solution, used for shaping ceramic parts through casting. The slurry can have carefully controlled viscosity and solid content to ensure proper flow, mold filling, and uniform particle packing. Once poured into a porous mold (usually made of plaster), the liquid phase is absorbed, leaving behind a consolidated ceramic shape that can be dried and later sintered.
Molding or casting: The slurry is poured into a porous mold (typically made of plaster), which begins absorbing the liquid. As the liquid is absorbed, solid zirconia particles accumulate along the inner surface of the mold, forming a dense ceramic layer. This process corresponds to cold isostatic pressing.
Drying: After casting, dry the wet mass slowly for about 10-30 days depending on thickness.
Pre-Sintering: Pre-sinter at 900-1050° C. using natural air sintering instead of hot isostatic pressure (HIP) sintering.
Post-Processing: Perform post-processing according to size and calculate shrinkage.

The process of making the zirconia green body can make it possible to reduce the shrinkage of the green body to 1.2100 or less during sintering. To create a zirconia ceramic with high translucency at a low temperature, the shrinkage can be 1.2100 or less. When the green body is sintered at a temperature between 1300-1400° C. or 1300-1450° C., the green body can achieve an average grain size of 200-500 nm and can be used to produce a dental restoration with high translucency.

Since the zirconia green body shrinks during sintering, the dental restoration is milled larger than the desired final size. The ratio by which the size is increased for milling is called the enlargement factor. It is also known as the shrinkage rate, which is the opposite concept of the enlargement factor. For example, if the final size is desired to be 1 mm after sintering, and the enlargement factor of the green body is 1.25, the green body should be milled to 1.25 mm. In this case, the shrinkage rate is 20% because 1.25 becomes 1.00.

When observing the sintered grains through Scanning Electron Microscope (SEM) images, the sintered grains are not completely round; some are oval-shaped, and most are irregular. There are various methods to measure the size of the sintered grain, but a simple and intuitive method can be used. The size of a particular sintered grain size can be defined as the longest distance between any two points on the boundary or perimeter of a specific grain.

When taking SEM photographs, a scale bar can be displayed at the bottom of the image, making it easy to determine the overall size. Additionally, the magnification factor can be provided, which helps in understanding the image comprehensively.

Table 1 shows the results of different zirconia samples of different enlargement factors subjected to various temperatures and sintering times. These samples are from dental discs currently available on the market (with thicknesses of 14 mm or 18 mm). The plates (14 mm×10 mm×2.0 mm) were milled out of the disc, and SEM images were taken of the 0.2 mm portion from the very top of the disc where the incisal/occlusal area is located. To measure the degree of wear on opposing teeth, the upper part of the zirconia blank where the occlusal or incisal area will be can be measured. Generally, the upper ¼ portion of the disc is considered the incisal/occlusal area in restorations. For a disc with a thickness of 12 mm, for example, the upper 3 mm portion can be considered the incisal/occlusal area.

The terms “upper” or “lower” portion of a zirconia disc or block do not refer to the physical orientation or geometric location within the raw material itself. Rather, they are used to indicate the intended anatomical positioning within a dental restoration.

Specifically, the “upper portion” corresponds to the area that will ultimately form the incisal or occlusal region of a crown or bridge. This region is often designed to have higher translucency, which is typically achieved by increasing the content of additives such as Y₂O₃ (yttria) or modifying the microstructure accordingly.

In contrast, the “lower portion” is aligned with the cervical or gingival area, where strength and opacity may be prioritized over translucency.

When fabricating a maxillary (upper) tooth restoration, the upper portion of the disc will face downwardly when placed in the mouth. Conversely, for a mandibular (lower) tooth, the upper portion of the disc will face upwardly in the patient's mouth.

It should be noted that when a zirconia disc or block is composed of a uniform, monolayer structure—i.e., without compositional or optical gradation across its thickness—the distinction between “upper” and “lower” portions becomes functionally insignificant.

In such cases, there is no variation in grain size, yttria concentration, or translucency between different regions of the disc, and the material exhibits consistent properties throughout. Therefore, references to “upper” or “lower” portions are generally not applicable or necessary for monolithic zirconia systems.

The zirconia discs/blocks used in these samples are all multilayer zirconia discs. A multilayer disc consists of an upper, middle, and lower layer, each with a chemically different composition. Generally, the upper layer uses 4Y or higher zirconia for aesthetic purposes. 4Y refers to 4 mol yttria-stabilized zirconia. The more stabilizer/additive, such as yttria, is used, the larger the grain size after sintering.

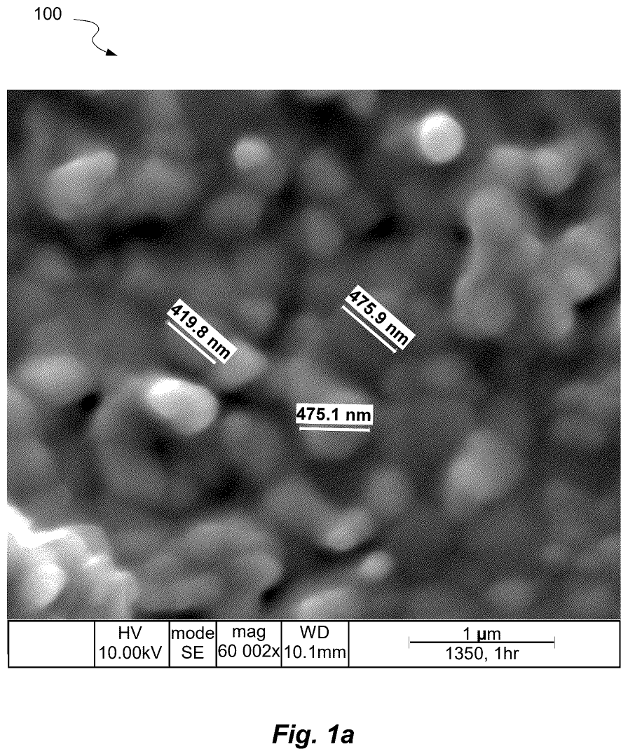

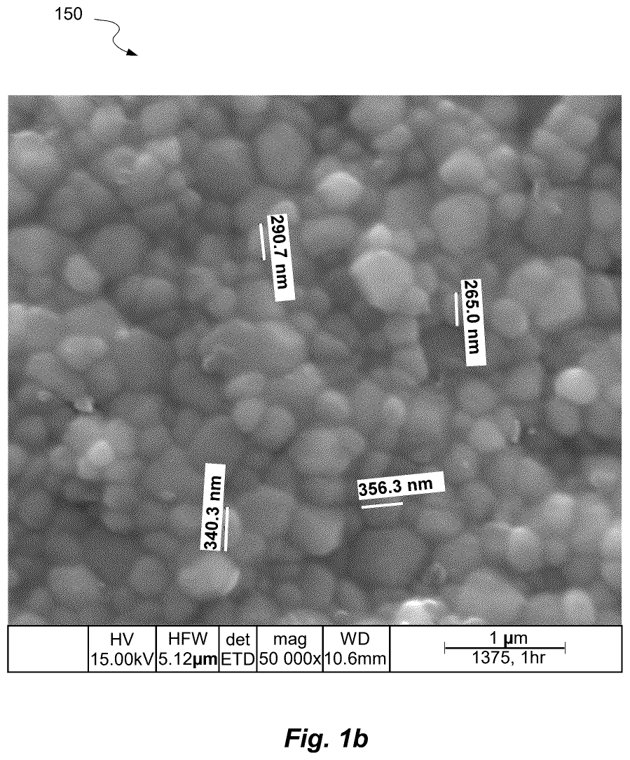

As shown in Table 1, Samples 1-6, which are made in accordance with the present technology, all have an enlargement factor not exceeding 1.2100 and were sintered at temperatures not exceeding 1400° C. As a result, none of the sintered grain sizes exceeded 1000 nm. These images correspond to FIGS. 1, 2, 3, and 4. However, Samples 7-12, which have a shrinkage factor exceeding 1.2100, were all sintered at temperatures above 1500° C. with a minimum modification, if needed, using each manufacturer's Instructions for Use (IFU). Their grain sizes all exceeded 1000 nm, resulting in larger sintered grain sizes. Sample number marked with asterisk (*) shows present technology.

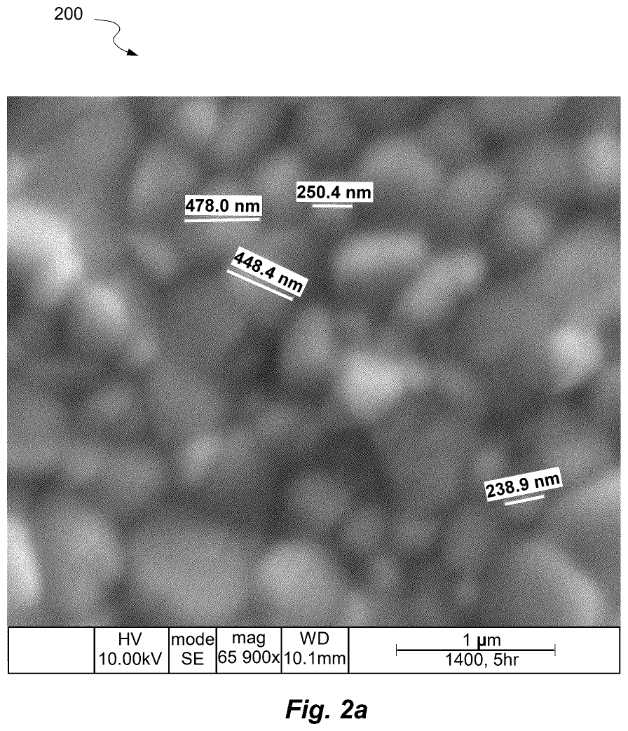

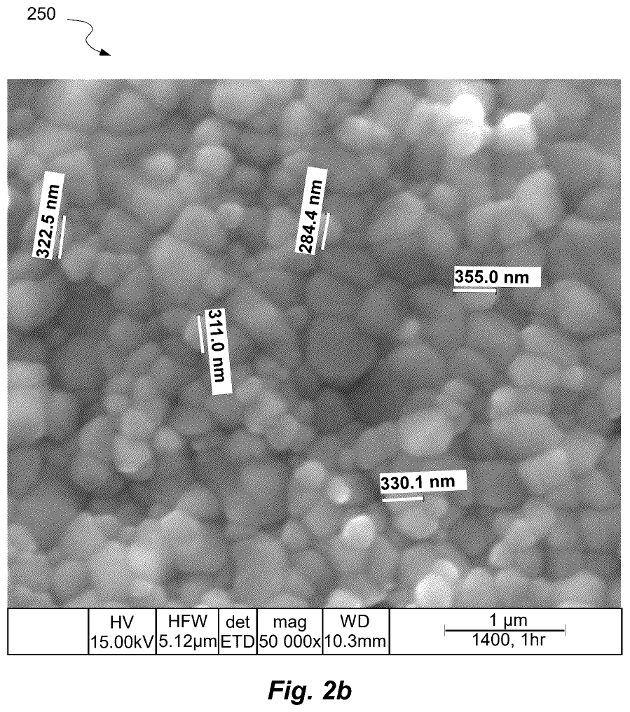

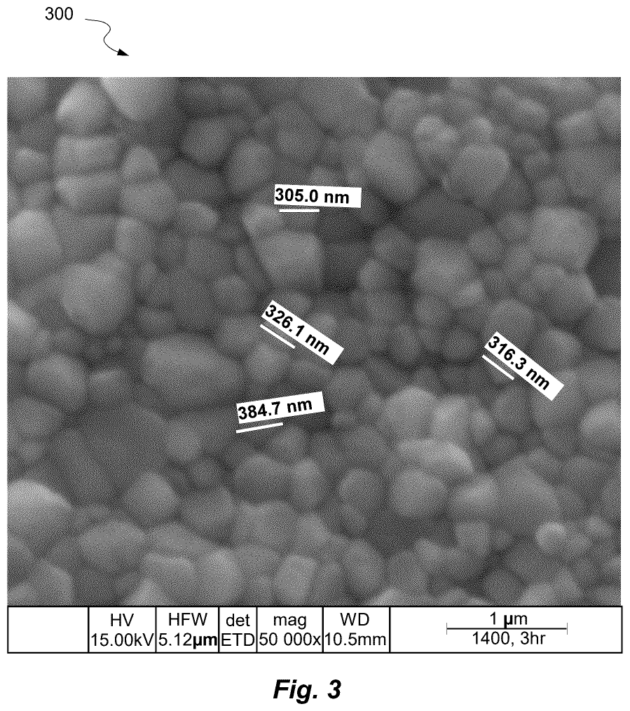

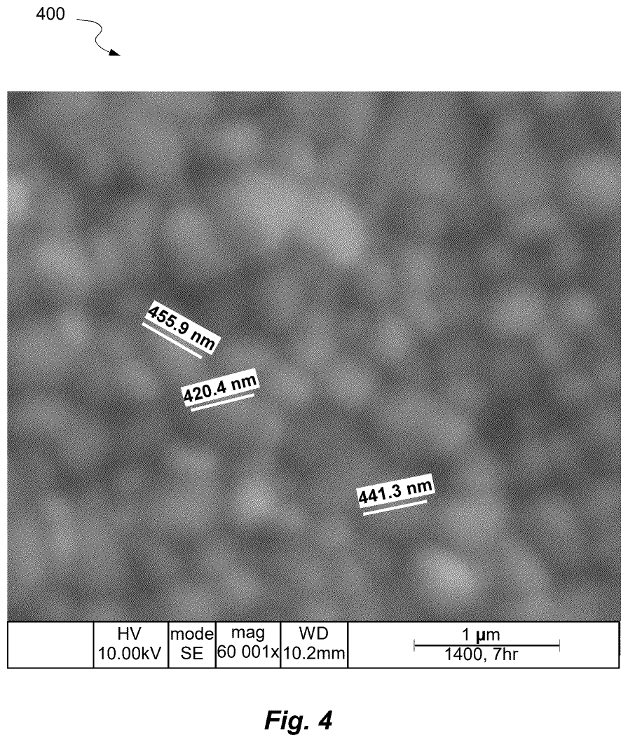

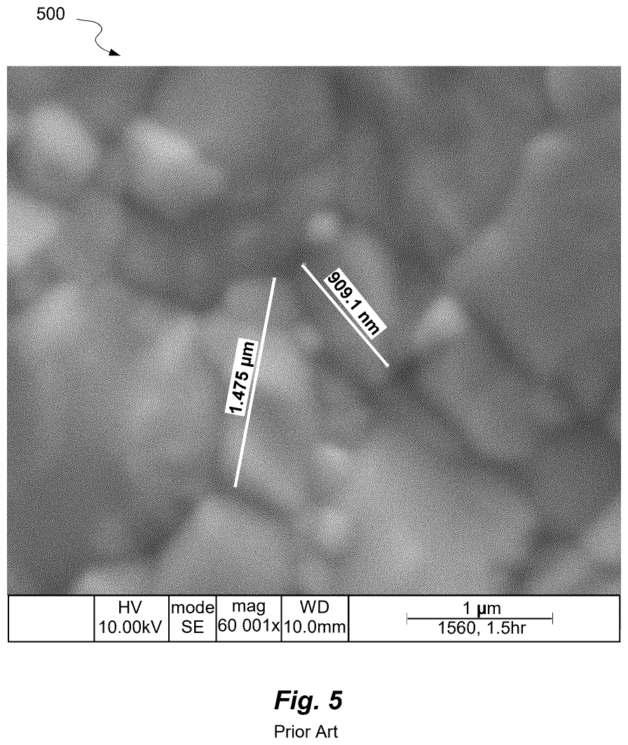

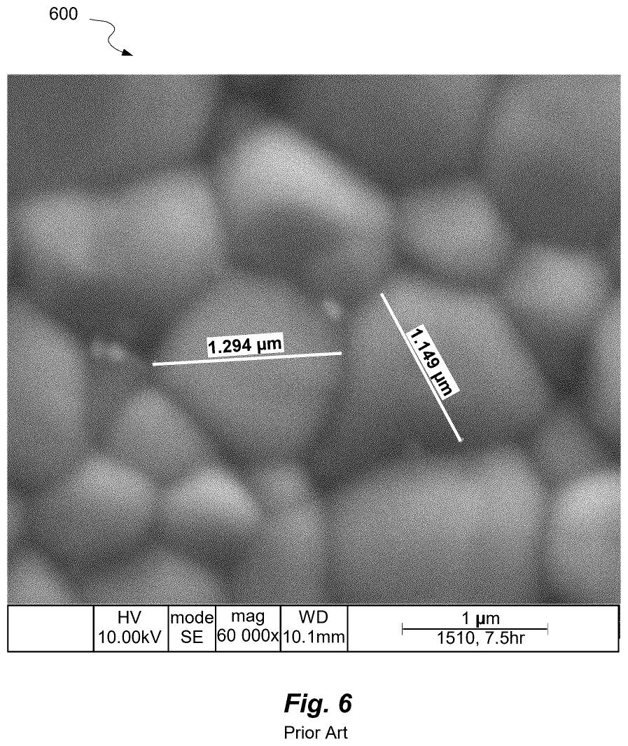

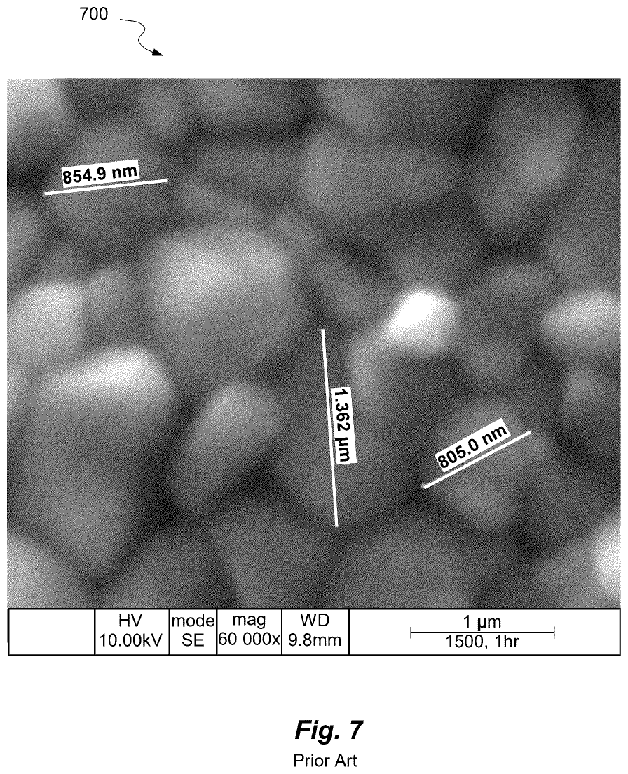

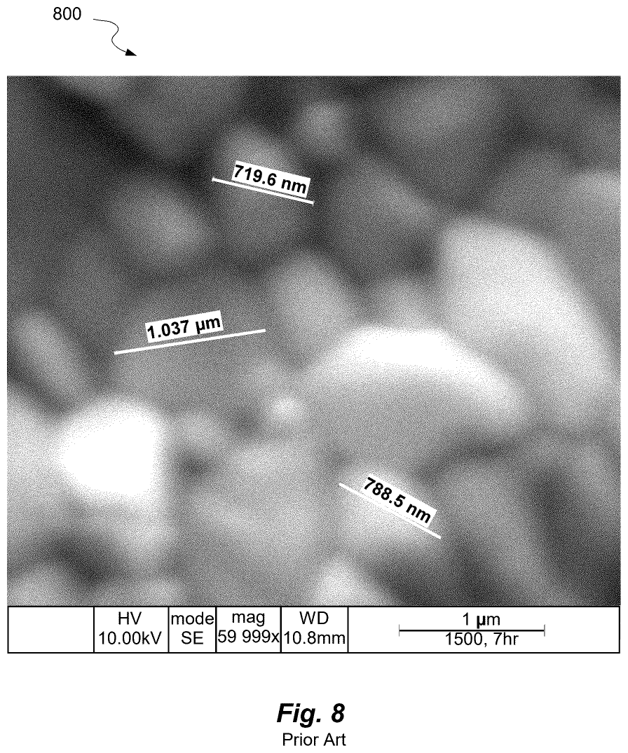

TABLE 1
Sintered grain size and L value for different
enlargement factor and sintering temperature

| | | | | | Grain size | | L value | |
|---|---|---|---|---|---|---|---|---|
| | Enlargement | Sintering | Sintered | | (sintered | | (at 2 mm | |
| Sample | factor | temperature | time | | body) | | thickness | |
| 1* | 1.2023 | 1350 | 1 | hour | 200-475 | nm | 78.1 | FIG. 1a |
| 2* | 1.2023 | 1375 | 1 | hour | 400-500 | nm | 79.2 | FIG. 1b |
| 3* | 1.2023 | 1400 | 5 | hours | 300-500 | nm | 80.3 | FIG. 2a |
| 4* | 1.2023 | 1400 | 1 | hour | 200-600 | nm | 79.5 | FIG. 2b |
| 5* | 1.2015 | 1400 | 3 | hours | 200-500 | nm | 78.9 | FIG. 3 |
| 6* | 1.2015 | 1400 | 7 | hours | 200-500 | nm | 80.1 | FIG. 4 |
| 7 | 1.2230 | 1560 | 1.5 | hours | 200-2000 | nm | 80.5 | FIG. 5 |
| 8 | 1.2319 | 1510 | 7.5 | hours | 500-1300 | nm | 79.4 | FIG. 6 |
| 9 | 1.2440 | 1500 | 1 | hour | 800-1400 | nm | 79.2 | FIG. 7 |
| 10 | 1.2450 | 1500 | 1 | hour | 700-1200 | nm | 79.8 | |
| 11 | 1.2450 | 1500 | 7 | hours | 800-1200 | nm | 80.4 | |
| 12 | 1.2440 | 1350 | 1 | hour | | | 14.7 | |
| 13 | 1.2450 | 1500 | 7 | hours | 700-1100 | nm | 80.5 | FIG. 8 |
| 14 | 1.2450 | 1350 | 1 | hour | | | 42.2 | |

There are various methods to measure translucency. Broadly, these include using a spectrophotometer to measure light transmission and measuring the contrast ratio. However, as used herein, the most well-known method in dentistry is used, which involves measuring light transmission with a spectrophotometer, specifically the VITA Easyshade®.

The present technology creates a zirconia green body with an enlargement factor of 1.2100 or less, which is then sintered at 1300-1400° C. It pertains to a green body with sintered grain sizes between 200-500 nm, ensuring that no grain exceeds 1000 nm. Until now, there has been no method to achieve a sintered body with an L value of at least 60 at a thickness of 2 mm, and a grain size between 200-500 nm by sintering within an hour at 1250-1400° C. or at 1350-1425° C.

When the sintering temperature is increased, the problem of the sintered grain size becoming larger is presented, especially for 4Y, 5Y or 6Y mol % yttria stabilized zirconia which is added on the incisal/occlusal area, which not only weakens the strength but also becomes abrasive to opposing teeth and is not beneficial for the patient.

Adding sintering aids such as yttria increases translucency, but there are two associated problems. First, the strength decreases, and secondly, the sintered grain size becomes larger (as shown in FIGS. 5-8), leading to increased abrasiveness to the opposing tooth.

The relationship between the sintering temperature and the resulting L values are as follows. The L value calculates the whiteness and blackness of the ceramic material, and at the same time measures the light transmission of the ceramic material to easily determine the quality of sintering. Using the VITA Easyshade® spectrophotometer, this can be easily measured. Table 2 below shows the L value measured after sintering a 14 mm Multilayer A2 shade disc with a thickness of 2 mm (14 mm×10 mm×2 mm) for one hour, selecting a point in the middle of the 14 mm height. The middle part of the sample represents the body color of a restoration. A higher L value indicates higher light transmission, but it does not necessarily mean the color is more accurate. As the temperature increases, the L value goes up, but the color in the restoration fades, reducing chroma and color accuracy. If the L value drops below 50, the restoration becomes too opaque to be used as a final restoration. By sintering the green body of the present technology, as described below, the following L values can be achieved. All samples were sintered in an hour. Sintering time within an hour means the time from the start until when the holding time at the highest temperature ends, as shown in the example sintering graph 900 in FIG. 9.

In relation to the current invention, sintering within an hour at a temperature of 1250° C. secures an L value of at least 54. Alternatively, sintering within an hour at a temperature of 1300° C. secures an L value of at least 74. Sintering within an hour at a temperature of 1350° C. secures an L value of at least 77. Sintering within an hour at temperature 1375° C. secures an L value of at least 78. Finally, sintering within an hour at temperatures 1400° C. secures an L value of at least 79, with the sintered grain size not exceeding 1000 nm.

TABLE 2
Sintering temperature (1 hour) and L value

| | Enlargement | Enlargement | Enlargement | Enlargement |
|---|---|---|---|---|
| | factor | factor | factor | factor |
| | 1.2023 | 1.2015 | 1.2450 | 1.2440 |
| 1550 | 81.8 | 80.5 | 80.8 | 80.5 |
| 1500 | 81.2 | 79.9 | 79.8 | 79.2 |
| 1450 | 80.1 | 78.7 | 77.7 | 76.9 |
| 1400 | 79.5 | 78.7 | 77.6 | 72.7 |
| 1375 | 79.2 | 77.6 | 61.7 | 13.3 |
| 1350 | 78.1 | 76.6 | 42.2 | 14.7 |
| 1300 | 75.9 | 73.5 | 15.0 | 11.7 |
| 1250 | 72.1 | 54.4 | 11.5 | 12.4 |

Lowering the sintering temperature can reduce the grain size. However, if the temperature is lowered below a certain level, there is a significant drop in translucency after sintering, especially when the enlargement factor is relatively higher. When translucency decreases, the dental restoration appears opaque and unnatural. Specifically, if the temperature is lowered to 1250° C., as shown in Table 2, translucency is not achieved at all, making it impossible to create aesthetically pleasing restorations.

Lowering the temperature as mentioned above can reduce the grain size, but it may also lower translucency. Increasing the amount of a sintering aid such as yttria can lower the sintering temperature to a certain level and increase translucency. However, too much yttria causes the grain size to become larger. There are already zirconia products on the market that use increased yttria for faster sintering, but the issue is that the grain size becomes significantly larger. This can lead to crack initiation and reduced strength. Regardless of yttria content, the goal of this invention, in part, is to achieve fast sintering while keeping the sintered grain size small.

In other words, because the green body is comparatively denser from the beginning, it has fewer residual pores even in the green state. Sintering such a green body at a low temperature (1250-1400° C.) can achieve high translucency and fast sintering.

The present technology can achieve the following characteristics, all in the same restoration:
Low-temperature sintering at 1250-1400° C.,
High translucency,
Multiple-case sintering, and
High strength.

Multiple-case sintering within an hour refers to the process of final sintering of at least 5, 10, 15, 20, 25, or 50 or more units of restorations simultaneously except for the frameworks for crown and bridge. The challenge of performing multiple case sintering with fast sintering within an hour lies in the heat energy required to fully sinter each restoration. As the number of restoration cases being sintered simultaneously increases, the amount of heat energy needed to achieve full sintering also increases. Conventional technology suggests that to sinter a large number of cases in a short time, higher temperatures must be used. Thus, other dental companies often recommend raising the temperature above 1500° C. However, raising the temperature, as discussed above, leads to the problem of larger grain sizes. When the grains become larger, the strength decreases, and the material becomes more abrasive in the patient's mouth, causing long-term damage to the opposing teeth.

The framework refers to the underlying substructure of a crown or multi-unit dental bridge restoration. It serves as a structural base over which veneering ceramics or other esthetic layers may be applied. Because the framework is ultimately covered with translucent porcelain powders—particularly in the incisal or occlusal area—it does not require intrinsic translucency or aesthetic properties. Therefore, even if the framework is sintered to a highly opaque and bright-white state, it does not negatively affect the final restoration. As such, multiple frameworks can be sintered rapidly—within one or two hours—without concern for optical quality.

According to experiments conducted in present technology, the extent to which the opposing tooth is worn down increases with grain size increase. In the experiment shown in Table 3, a load of 5 kg (comparable to 49 N of chewing force) was applied, and 1.2 million mastication cycles were performed. The result was the larger the sintered grain size, the more damage was left on the opposing tooth. The opposing tooth damage and sintered grain size in this experiment represent approximate average values based on visual observation of SEM images.

TABLE 3
Sintered grain size negatively affecting the opposing tooth

| Sample | Sintered grain size | | Opposing tooth damage | |
|---|---|---|---|---|
| Sample 1 | 200-500 | nm | 500 | nm |
| Sample 2 | 200-2000 | nm | 2000 | nm |
| Sample 3 | 500-1300 | nm | 700 | nm |
| Sample 4 | 700-2000 | nm | 1300 | nm |

Reducing the sintered grain size of zirconia, especially under 1,000 nm, preferably with average size of 200-500 nm, improves the uniformity and efficiency of the sintering process, which directly contributes to the ability to sinter a larger number of crowns simultaneously. This may occur for several reasons. First, improved thermal homogeneity—finer grains promote more uniform heat distribution and faster thermal equilibration throughout each unit. As a result, temperature gradients across multiple crowns are minimized, reducing the risk of uneven shrinkage or warping. Second, reduced internal stress and distortion—smaller grain size leads to more controlled densification behavior with lower internal stresses during sintering. This allows multiple crowns to be sintered in close proximity without causing deformation, chipping, or contact damage. Third, faster and more predictable shrinkage—fine-grained zirconia shrinks more evenly and predictably. This uniform shrinkage enables tighter packing of restorations in the sintering tray while maintaining dimensional stability and fit accuracy. Fourth, low risk of grain growth-related defects—in large batch sintering, thermal variation can lead to exaggerated grain growth in some units. Fine starting grains are less prone to abnormal grain growth, improving consistency across all crowns in a full load.

Thus, by maintaining the sintered grain size below 1,000 nm, preferably with average size of 200-500 nm, thermal shrinkage occurs more uniformly across units, thereby minimizing distortion, cracking, and inter-unit interference during sintering. As a result, the zirconia material enables simultaneous sintering of a significantly greater number of dental restorations—such as crowns or bridges—within a single furnace cycle, without compromising dimensional accuracy or mechanical integrity. Specifically, sintering at least 5, 10, 15, 20, 25, 30, 40 or 50 units of restorations simultaneously can be possible within an hour of sintering time. A zirconia green body that is sinterable to produce at least 20 units of dental restorations within one hour under standard dental laboratory sintering conditions would be very beneficial.

Unlike prior art fast-sintering zirconia products, which are often limited to single-unit restorations or those with a wall thickness of less than 3 mm, the zirconia green body of the present technology enables the sintering of multiple restorations (e.g., 20 or more units) in a single batch within one hour, without compromising translucency or mechanical strength. This is achieved without relying on specialized equipment such as HIP furnaces or inert gas environments, and under standard atmospheric sintering conditions commonly found in dental laboratories.

The present technology overcomes the microstructural limitations of previous materials, which typically exhibit irregular grain arrangements and oversized grains exceeding 1,000 nanometers. These structural shortcomings have historically led to trapped porosity, increased optical opacity, and restricted throughput. In contrast, the present technology allows for consistent, high-volume production with excellent esthetic and mechanical properties.

The zirconia green body of the present technology is uniquely designed to produce a sintered structure in which the majority of grain sizes are confined to the range of 200 to 500 nanometers, or in some embodiments, 200 to 600 nanometers or 200 to 700 nanometers, with no grains exceeding 1,000 nanometers in diameter. Unlike conventional zirconia materials, which often contain a significant proportion of large, irregular grains of over 1 micron, the present technology ensures a highly homogeneous grain structure throughout the sintered body.

This uniform and fine-grained microstructure leads to a markedly more efficient and evenly distributed heat transfer during the sintering process. As a result, the green body can be fully sintered within one hour, and in certain optimized conditions, within 30 minutes, without compromising mechanical properties or dimensional integrity.

In practical terms, this sintering efficiency allows for the fabrication of at least 5, 10, 15, 20, 25, or even 50 units of dental restorations in a single sintering cycle. These restorations may include single crowns, multi-unit bridges, or full-arch All-on-X implant-supported prostheses. The ability to sinter such high-throughput cases in reduced time stems directly from the microstructural advantages of the present technology, particularly its controlled and submicron-limited sintered grain size distribution, as shown in Table 4.

TABLE 4
Comparison between conventional zirconia and present technology

| | Conventional | | |
|---|---|---|---|
| Feature | Zirconia | | Present Technology |
| Max Grain Size | >1,000 | nm | ≤1,000 nm (no |
| | | | grains >1,000 nm) |
| Majority Grain Range | 500-1,500 | nm | 200-500 nm (or up to |
| | | | 700 nm) |
| Grain Uniformity | Heterogeneous | | Highly Homogeneous |
| Sintering Time | ≥2 | hours | ≤2 hours (or 1 hour or |
| Required | | | even 30 min) |
| Simultaneous Units | 1-3 | units | ≥5, 10, 15, 20 or 50 |
| Sinterable | | | units |

### Example 1: Sintering of a Full-Arch All-on-X Restoration

A full-arch zirconia prosthesis was designed for an edentulous patient using a digital All-on-X protocol. The prosthesis design included 14 individual tooth units (from second molar to second molar), monolithically connected to form a single fixed restoration. The green body was milled from a multilayer zirconia disc fabricated according to the present invention.

The sintering was performed under standard conditions (1,400° C. for 1 hour) in ambient air using a conventional dental laboratory furnace. The full-arch restoration (considered a single case comprising 14 units) was sintered alongside 16 additional single crowns, totaling 30 units in the same sintering cycle.

Post-sintering analysis showed:
No visible warping or marginal distortion;
Uniform shrinkage with accurate fit;
Flexural strength >1000 MPa (per ISO 6872:2015, bar-shaped specimen cut from body region); and
Translucency consistent with anterior esthetic restorations.

Thus, even a large, complex restoration, such as an All-on-X full-arch prosthesis, can be sintered in one hour alongside additional units without sacrificing quality or performance.

The present technology demonstrates not only the ability to sinter individual crowns or small bridges rapidly, but also a suitability for high-unit, full-arch restorations such as All-on-X cases. As shown in Example 1, a 14-unit full-arch restoration was successfully sintered in one hour under standard laboratory conditions, together with 16 additional crowns−totaling 30 units in a single cycle. No distortion, color gradient anomalies, or loss in mechanical strength were observed.

This example illustrates that the present zirconia green body enables reliable, high-throughput fast sintering, even for restorations previously considered incompatible with one-hour sintering protocols due to their size, complexity, or esthetic demands.

Two identical All-on-X cases, each consisting of a 14-unit full-arch restoration, were sintered within one or two hours, and comparable results were consistently achieved. When sintering was performed at 1,400° C. for 2 hours, there was a slight improvement in aesthetic characteristics.

### Example 2: Sintering of Two Full-Arch All-on-X Restorations in a Two-Hour Cycle

Two full-arch zirconia prostheses (All-on-X cases) were prepared for this experiment. Each prosthesis was a 14-unit implant-supported bridge spanning from the second molar to the second molar, designed using a digital All-on-X protocol. The green bodies of both full-arch restorations were milled from a multilayer zirconia disc according to the process described in the present technology. Both cases (totaling 28 units of dental restoration) were placed together in a single sintering tray equipped with support walls to stabilize the structures during firing.

Sintering was performed in a conventional dental furnace at 1,425° C. for a holding time of 30 minutes (cooling time not included). Upon completion of the 2-hour sintering time, the furnace and the restorations were allowed to cool naturally to ambient temperature.

Post-sintering analysis of the two All-on-X prostheses showed: no visible warping or marginal distortion in either restoration; uniform shrinkage with accurate fit on their corresponding models; an average zirconia grain size (including in the incisal areas) not exceeding 1,000 nm; and flexural strength values comparable to those of the 1-hour sintered case (exceeding 1000 MPa per ISO 6872:2015, measured on bar specimens). The optical properties were also favorable, with a slight improvement in translucency observed relative to the one-hour sintered restoration. Accordingly, this experiment demonstrates that two full-arch All-on-X zirconia restorations can be successfully co-sintered within a two-hour sintering time without compromising their structural integrity or esthetic quality.

### Example 3: Sintering of Four Full-Arch All-on-X Restorations Using Two Stacked Trays

Four full-arch zirconia prostheses (All-on-X cases) were prepared using the same protocol as described in Example 2. Each prosthesis was a 14-unit monolithic implant-supported bridge spanning from second molar to second molar. Two restorations were placed in a single sintering tray with wall supports. An identical second tray containing two additional restorations was stacked on top of the first tray. All four restorations (totaling 56 units of dental restorations) were sintered together in one cycle.

Sintering was performed under the same standard conditions (1,425° C. for 2 hours in ambient air), followed by natural cooling.

Post-sintering evaluation revealed no observable warping or distortion in any of the four restorations. Shrinkage remained uniform with clinically accurate fit, and all sintered grain sizes, including those in the incisal regions, were confirmed to be under 1,000 nm. Flexural strength measurements were consistent with the results obtained in Example 2 (exceeding 1,000 MPa), and translucency characteristics remained favorable.

This example confirms that four full-arch All-on-X zirconia prostheses can be successfully sintered simultaneously within a two-hour sintering time, using a stacked tray configuration, without compromising structural or esthetic outcomes.

In multilayer zirconia systems, it is common to increase the concentration of yttria (Y₂O₃) in the upper portion of the disc or block in order to enhance translucency in the incisal or occlusal region of the final dental restoration. In the context of a zirconia disc, the term “incisal area” refers to the uppermost 3-4 mm region of the disc, particularly in a disc with a thickness of approximately 14 mm. This region corresponds to the incisal or occlusal zone of the final dental restoration. To mimic the natural translucency of human enamel, the incisal area is typically formulated with a higher concentration of additives, such as Yttria (Y₂O₃), in order to enhance optical translucency. While this approach effectively improves optical properties, it inherently leads to an increase in sintered grain size, which in turn causes a reduction in flexural strength due to grain boundary weakening and enhanced crack propagation.

However, in the present invention, the green body is engineered with a nano size particle size (200-500 nm) prior to sintering, which serves to limit excessive grain growth—even in regions with higher yttria content. As a result, the sintered body maintains a flexural strength of over 800 MPa, and preferably 900 MPa or higher, despite increased translucency in the upper layer as shown in Table 5.

TABLE 5

| | | | | Temper- | | Holding | | | | | Strength − |
|---|---|---|---|---|---|---|---|---|---|---|---|
| | | | Temperature | ature | Sintering | time | | Sintering | Average | Grains | incisal |
| | Y2O3 | Y2O3 | raising rate | raising rate | Temper- | (Retention | | time | grain | exceeding | area |
| Sample | (wt %) | (Mol %) | (° C./min) | (° C./hr) | ature | time) | | (hour) | size (nm) | 1,000 nm | (Mpa) |
| Sample | 8.8 | 5 | 150° C./min to | 9000° C./hr | 1400 | 10 | min | 1 | 200-500 | None | 942 |
| 1 | | | 900° C. 12° C./ | to 900° C.; | | | | | | | |
| | | | min to 1400° C. | 720° C./hr | | | | | | | |
| | | | | to 1400° C. | | | | | | | |
| Sample | 8.8 | 5 | 150° C./min to | 9000° C./hr | 1400 | 30 | min | 2 | 200-500 | None | 923 |
| 2 | | | 900° C. 6° C./ | to 900° C.; | | | | | | | |
| | | | min to 1400° C. | 360° C./hr | | | | | | | |
| | | | | to 1400° C. | | | | | | | |
| Sample | 9.6 | 5.5 | 150° C./min to | 9000° C./hr | 1400 | 10 | min | 1 | 200-500 | None | 898 |
| 3 | | | 900° C. 12° C./ | to 900° C.; | | | | | | | |
| | | | min to 1400° C. | 720° C./hr | | | | | | | |
| | | | | to 1400° C. | | | | | | | |
| Sample | 9.6 | 5.5 | 150° C./min to | 9000° C./hr | 1400 | 30 | min | 2 | 200-500 | None | 871 |
| 4 | | | 900° C. 6° C./ | to 900° C.; | | | | | | | |
| | | | min to 1400° C. | 360° C./hr | | | | | | | |
| | | | | to 1400° C. | | | | | | | |

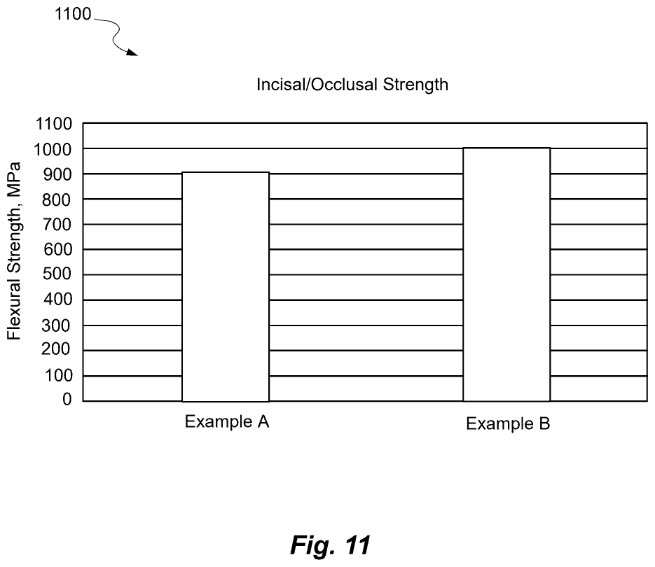

Referring to FIG. 11, an example graph 1100 of the incisal/occlusal strengths of dental restorations, according to the present technology. A first example, Example A, has an incisal/occlusal flexural strength of 900 mega Pascals (MPa), while a second example, Example B, has an incisal/occlusal flexural strength of 1000 MPa.

The enhanced mechanical performance may be attributed to:
Grain boundary strengthening: Smaller grain size (around 200-500 nm) increases the number of grain boundaries, which act as barriers to crack propagation, thereby enhancing overall fracture resistance.
Inhibition of abnormal grain growth: A finer starting powder reduces the driving force for uncontrolled grain coarsening during sintering, particularly in high-yttria zones.
Improved stress distribution: Uniform, fine-grained microstructures help distribute thermal and mechanical stress more evenly, reducing the risk of localized failure.

Accordingly, the present technology provides a multilayer zirconia material that balances high translucency with mechanical robustness, suitable for both aesthetic and load-bearing dental restorations.

Further aspects of the invention include:
1. A dental zirconia-based green body, which optionally includes one or more features of any one of more of the preceding and/or following aspects, comprising: a zirconium oxide and at least one other non-zirconium oxide; the at least one other non-zirconium oxide equaling 7-20 weight percent (wt %) based on a total weight percent of the zirconia-based green body; the zirconia-based green body being sinterable to form a sintered body with sintered grains; and wherein all the sintered grains of the sintered body have a sintered grain size less than 1000 nano-meters (nm); wherein a majority of the sintered grains of the sintered body have a sintered grain size greater than 100 nm.
2. A dental zirconia-based green body, which optionally includes one or more features of any one of more of the preceding and/or following aspects, wherein at least 70% of the sintered grains of the sintered body have a sintered grain size of 200-500 nm.
3. A dental zirconia-based green body, which optionally includes one or more features of any one of more of the preceding and/or following aspects, wherein at least 50% of the sintered grains of the sintered body have a sintered grain size of 200-500 nm.
4. A dental zirconia-based green body, which optionally includes one or more features of any one of more of the preceding and/or following aspects, wherein the dental zirconia-based green body is simultaneously sinterable to form at least five units of dental restorations within one hour of sintering time and in ambient air without atmospheric control or hot isostatic pressure (HIP).
5. A dental zirconia-based green body, which optionally includes one or more features of any one of more of the preceding and/or following aspects, wherein the at least five units of dental restorations further comprise at least five of a single-unit crown, a multi-unit bridge, a full-arch All-on-X implant-supported prostheses, or combinations thereof.
6. A dental zirconia-based green body, which optionally includes one or more features of any one of more of the preceding and/or following aspects, wherein the zirconia-based green body is sinterable at a temperature of 1,350 to 1,425 degrees Celsius (° C.) to form the sintered body with the sintered grains.
7. A dental zirconia-based green body, which optionally includes one or more features of any one of more of the preceding and/or following aspects, wherein: the sintered body has an incisal or occlusal part corresponding to an incisal or occlusal area of a dental restoration; and the incisal or occlusal part of the sintered body having a flexural strength of at least 800 mega-Pascals (MPa).
8. A dental zirconia-based green body, which optionally includes one or more features of any one of more of the preceding and/or following aspects, comprising: a zirconium oxide and at least one other non-zirconium oxide; the at least one other non-zirconium oxide equaling 7-20 weight percent (wt %) based on a total weight percent of the zirconia-based green body; the zirconia-based green body being sinterable at a temperature of 1,350 to 1,425 degrees Celsius (° C.) to form a sintered body with sintered grains; and wherein all the sintered grains of the sintered body have a sintered grain size less than 1000 nano-meters (nm); wherein a majority of the sintered grains of the sintered body have a sintered grain size greater than 100 nm.
9. A dental zirconia-based green body, which optionally includes one or more features of any one of more of the preceding and/or following aspects, wherein: the sintered body has an incisal or occlusal part corresponding to an incisal or occlusal area of a dental restoration; and the incisal or occlusal part of the sintered body having a flexural strength of at least 800 mega-Pascals (MPa).
10. A dental zirconia-based green body, which optionally includes one or more features of any one of more of the preceding and/or following aspects, wherein at least 70% of the sintered grains of the sintered body have a sintered grain size of 200-500 nm.
11. A dental zirconia-based green body, which optionally includes one or more features of any one of more of the preceding and/or following aspects, wherein at least 50% of the sintered grains of the sintered body have a sintered grain size of 200-500 nm.
12. A dental zirconia-based green body, which optionally includes one or more features of any one of more of the preceding and/or following aspects, wherein the dental zirconia-based green body is sized to provide milling at least five separate green-body dental restorations from the zirconia-based green body; and wherein the at least five separate green-body dental restorations are sinterable simultaneously in less than one hour to obtain at least five finished dental restorations.
13. A dental zirconia-based green body, which optionally includes one or more features of any one of more of the preceding and/or following aspects, wherein the at least five finished dental restorations further comprise at least five of a single-unit crown, a multi-unit bridge, a full-arch All-on-X implant-supported prostheses, or combinations thereof.
14. A dental zirconia-based green body, which optionally includes one or more features of any one of more of the preceding and/or following aspects, comprising: a zirconium oxide and at least one other non-zirconium oxide; the at least one other non-zirconium oxide equaling 8.8-20 weight percent (wt %) based on a total weight percent of the zirconia-based green body; and the zirconia-based green body being sinterable to form a sintered body with an incisal or occlusal part corresponding to an incisal or occlusal area of a dental restoration; wherein the incisal or occlusal part of the sintered body having a flexural strength greater than 800 mega-Pascals (MPa).
15. A dental zirconia-based green body, which optionally includes one or more features of any one of more of the preceding and/or following aspects, wherein: the zirconia-based green body is sinterable to form the sintered body with sintered grains; wherein all the sintered grains of the sintered body have a sintered grain size less than 1000 nano-meters (nm).
16. A dental zirconia-based green body, which optionally includes one or more features of any one of more of the preceding and/or following aspects, wherein at least 70% of the sintered grains of the sintered body have a sintered grain size of 200-500 nm.
17. A dental zirconia-based green body, which optionally includes one or more features of any one of more of the preceding and/or following aspects, wherein at least 50% of the sintered grains of the sintered body have a sintered grain size of 200-500 nm.
18. A dental zirconia-based green body, which optionally includes one or more features of any one of more of the preceding and/or following aspects, wherein the dental zirconia-based green body is sized to provide milling at least five separate green-body dental restorations from the zirconia-based green body; and wherein the at least five separate green-body dental restorations are sinterable simultaneously in less than one hour to obtain at least five finished dental restorations.
19. A dental zirconia-based green body, which optionally includes one or more features of any one of more of the preceding and/or following aspects, wherein the at least five finished dental restorations further comprise at least five of a single-unit crown, a multi-unit bridge, a full-arch All-on-X implant-supported prostheses, or combinations thereof.
20. A dental zirconia-based green body, which optionally includes one or more features of any one of more of the preceding and/or following aspects, wherein the zirconia-based green body is sinterable at a temperature of 1,350 to 1,425 degrees Celsius (° C.) to form the sintered body.
21. A dental zirconia-based green body, which optionally includes one or more features of any one of more of the preceding and/or following aspects, comprising: a zirconium oxide and at least one other non-zirconium oxide; and the zirconia-based green body being simultaneously sinterable to form at least five units of dental restorations within one hour of sintering time and in ambient air without atmospheric control or hot isostatic pressure (HIP).
22. A dental zirconia-based green body, which optionally includes one or more features of any one of more of the preceding and/or following aspects, further comprising the zirconia-based green body being simultaneously sinterable to form at least ten units of dental restorations within one hour of sintering time.
23. A dental zirconia-based green body, which optionally includes one or more features of any one of more of the preceding and/or following aspects, further comprising the zirconia-based green body being simultaneously sinterable to form at least twenty units of dental restorations within one hour of sintering time.
24. A dental zirconia-based green body, which optionally includes one or more features of any one of more of the preceding and/or following aspects, wherein the at least five units of dental restorations further comprise at least five of a single-unit crown, a multi-unit bridge, a full-arch All-on-X implant-supported prostheses, or combinations thereof.
25. A dental zirconia-based green body, which optionally includes one or more features of any one of more of the preceding and/or following aspects, wherein: the at least one other non-zirconium oxide equaling 7-20 weight percent (wt %) based on a total weight percent of the zirconia-based green body; the zirconia-based green body being sinterable to form a sintered body with sintered grains; and wherein all the sintered grains of the sintered body have a sintered grain size less than 1000 nano-meters (nm); wherein a majority of the sintered grains of the sintered body have a sintered grain size greater than 100 nm.
26. A dental zirconia-based green body, which optionally includes one or more features of any one of more of the preceding and/or following aspects, wherein: the at least one other non-zirconium oxide equaling 7-20 weight percent (wt %) based on a total weight percent of the zirconia-based green body; the zirconia-based green body being sinterable at a temperature of 1,350 to 1,425 degrees Celsius (° C.) to form a sintered body with sintered grains; and wherein all the sintered grains of the sintered body have a sintered grain size less than 1000 nano-meters (nm); wherein a majority of the sintered grains of the sintered body have a sintered grain size greater than 100 nm.
27. A dental zirconia-based green body, which optionally includes one or more features of any one of more of the preceding and/or following aspects, wherein: the at least one other non-zirconium oxide equaling 7-20 weight percent (wt %) based on a total weight percent of the zirconia-based green body; and the zirconia-based green body being sinterable to form a sintered body with an incisal or occlusal part corresponding to an incisal or occlusal area of a dental restoration; wherein the incisal or occlusal part of the sintered body having a flexural strength of at least 800 mega-Pascals (MPa).
28. A dental zirconia-based green body, which optionally includes one or more features of any one of more of the preceding and/or following aspects, comprising: a zirconium oxide and at least one other non-zirconium oxide; and the zirconia-based green body being simultaneously sinterable to form at least ten units of dental restorations, except for a framework for a crown or a bridge, within 90 minutes of sintering time and in ambient air without atmospheric control or hot isostatic pressure (HIP).
29. A dental zirconia-based green body, which optionally includes one or more features of any one of more of the preceding and/or following aspects, further comprising the zirconia-based green body being simultaneously sinterable to form at least twenty units of dental restorations, except for the framework for the crown or the bridge, within 90 minutes of sintering time.
30. A dental zirconia-based green body, which optionally includes one or more features of any one of more of the preceding and/or following aspects, wherein the at least ten units of dental restorations further comprise at least ten of a single-unit crown, a multi-unit bridge, a full-arch All-on-X implant-supported prostheses, or combinations thereof, except for the framework for the crown or the bridge.
31. A dental zirconia-based green body, which optionally includes one or more features of any one of more of the preceding and/or following aspects, comprising: a zirconium oxide and at least one other non-zirconium oxide; and the zirconia-based green body being sinterable to form at least one All-on-X dental restoration within two hours of sintering time and in ambient air without atmospheric control or hot isostatic pressure (HIP).
32. A dental zirconia-based green body, which optionally includes one or more features of any one of more of the preceding and/or following aspects, further comprising: the at least one other non-zirconium oxide equaling 8.8-20 weight percent (wt %) based on a total weight percent of the zirconia-based green body; and the zirconia-based green body being sinterable to form a sintered body with an incisal or occlusal part corresponding to an incisal or occlusal area of a dental restoration; wherein the incisal or occlusal part of the sintered body having a flexural strength greater than 800 mega-Pascals (MPa).
33. A method of producing dental restorations, which optionally includes one or more features of any one of more of the preceding and/or following aspects, comprising: obtaining a zirconia-based green body; milling at least five separate green-body dental restorations from the zirconia-based green body; and sintering the at least five separate green-body dental restorations simultaneously in less than one hour to obtain at least five finished dental restorations.
34. A method of producing dental restorations, which optionally includes one or more features of any one of more of the preceding and/or following aspects, wherein the at least five separate green-body dental restorations further comprises at least ten separate green-body dental restorations; and wherein the at least five finished dental restorations further comprises at least ten finished dental restorations.
35. A method of producing dental restorations, which optionally includes one or more features of any one of more of the preceding and/or following aspects, wherein the at least five separate green-body dental restorations further comprises at least twenty separate green-body dental restorations; and wherein the at least five finished dental restorations further comprises at least twenty finished dental restorations.
36. A method of producing dental restorations, which optionally includes one or more features of any one of more of the preceding and/or following aspects, wherein the at least five finished dental restorations further comprise at least five of a single-unit crown, a multi-unit bridge, a full-arch All-on-X implant-supported prostheses, or combinations thereof.
37. A method of producing dental restorations, which optionally includes one or more features of any one of more of the preceding and/or following aspects, wherein the at least five finished dental restorations further comprise at least one full-arch All-on-X implant-supported prostheses.

The foregoing description conveys the best understanding of the objectives and advantages of the present invention. Different embodiments may be made of the inventive concept of this invention. It is to be understood that all matter disclosed herein is to be interpreted merely as illustrative, and not in a limiting sense.

Reference throughout this specification to “an example” or “exemplary” means that a particular feature, structure, or characteristic described in connection with the example is included in at least one embodiment of the present invention. Thus, appearances of the phrases “in an example” or the word “exemplary” in various places throughout this specification are not necessarily all referring to the same embodiment.

As used herein, a plurality of items, structural elements, compositional elements, and/or materials can be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention can be referred to herein along with alternatives for the various components thereof. It is understood that such embodiments, examples, and alternatives are not to be construed as defacto equivalents of one another, but are to be considered as separate and autonomous representations of the present invention.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

What is claimed is:

1. A dental zirconia-based green body, comprising:
a zirconium oxide and at least one other non-zirconium oxide;
the at least one other non-zirconium oxide equaling 7-20 weight percent (wt %) based on a total weight percent of the zirconia-based green body;
the zirconia-based green body being sinterable to form a sintered body with sintered grains; and
wherein all the sintered grains of the sintered body have a sintered grain size less than 1000 nano-meters (nm);
wherein a majority of the sintered grains of the sintered body have a sintered grain size greater than 100 nm.

2. The dental zirconia-based green body of claim 1, wherein the dental zirconia-based green body is simultaneously sinterable to form at least five units of dental restorations within one hour of sintering time and in ambient air without atmospheric control or hot isostatic pressure (HIP).

3. The dental zirconia-based green body of claim 2, wherein the at least five units of dental restorations further comprise at least five of a single-unit crown, a multi-unit bridge, a full-arch All-on-X implant-supported prostheses, or combinations thereof.

4. The dental zirconia-based green body of claim 1, wherein the zirconia-based green body is sinterable at a temperature of 1,350 to 1,425 degrees Celsius (° C.) to form the sintered body with the sintered grains.

5. The dental zirconia-based green body of claim 1, wherein:
the sintered body has an incisal or occlusal part corresponding to an incisal or occlusal area of a dental restoration; and
the incisal or occlusal part of the sintered body having a flexural strength of at least 800 mega-Pascals (MPa).

6. A dental zirconia-based green body, comprising:
a zirconium oxide and at least one other non-zirconium oxide;
the at least one other non-zirconium oxide equaling 7-20 weight percent (wt %) based on a total weight percent of the zirconia-based green body;
the zirconia-based green body being sinterable at a temperature of 1,350 to 1,425 degrees Celsius (° C.) to form a sintered body with sintered grains; and
wherein all the sintered grains of the sintered body have a sintered grain size less than 1000 nano-meters (nm);
wherein a majority of the sintered grains of the sintered body have a sintered grain size greater than 100 nm.

7. The dental zirconia-based green body of claim 6, wherein:
the sintered body has an incisal or occlusal part corresponding to an incisal or occlusal area of a dental restoration; and
the incisal or occlusal part of the sintered body having a flexural strength of at least 800 mega-Pascals (MPa).

8. The dental zirconia-based green body of claim 6, wherein the dental zirconia-based green body is sized to provide milling at least five separate green-body dental restorations from the zirconia-based green body; and wherein the at least five separate green-body dental restorations are sinterable simultaneously in less than one hour to obtain at least five finished dental restorations.

9. The dental zirconia-based green body of claim 8, wherein the at least five finished dental restorations further comprise at least five of a single-unit crown, a multi-unit bridge, a full-arch All-on-X implant-supported prostheses, or combinations thereof.

10. A dental zirconia-based green body, comprising:
a zirconium oxide and at least one other non-zirconium oxide;
the at least one other non-zirconium oxide equaling 8.8-20 weight percent (wt %) based on a total weight percent of the zirconia-based green body; and
the zirconia-based green body being sinterable to form a sintered body with an incisal or occlusal part corresponding to an incisal or occlusal area of a dental restoration;
wherein the incisal or occlusal part of the sintered body having a flexural strength greater than 800 mega-Pascals (MPa).

11. The dental zirconia-based green body of claim 10, wherein:
the zirconia-based green body is sinterable to form the sintered body with sintered grains;
wherein all the sintered grains of the sintered body have a sintered grain size less than 1000 nano-meters (nm).

12. The dental zirconia-based green body of claim 10, wherein the dental zirconia-based green body is sized to provide milling at least five separate green-body dental restorations from the zirconia-based green body; and wherein the at least five separate green-body dental restorations are sinterable simultaneously in less than one hour to obtain at least five finished dental restorations.

13. The dental zirconia-based green body of claim 12, wherein the at least five finished dental restorations further comprise at least five of a single-unit crown, a multi-unit bridge, a full-arch All-on-X implant-supported prostheses, or combinations thereof.

14. The dental zirconia-based green body of claim 10, wherein the zirconia-based green body is sinterable at a temperature of 1,350 to 1,425 degrees Celsius (° C.) to form the sintered body.

15. A dental zirconia-based green body, comprising:
a zirconium oxide and at least one other non-zirconium oxide; and
the zirconia-based green body being simultaneously sinterable to form at least five units of dental restorations within one hour of sintering time and in ambient air without atmospheric control or hot isostatic pressure (HIP).

16. The dental zirconia-based green body of claim 15, further comprising the zirconia-based green body being simultaneously sinterable to form at least ten units of dental restorations within one hour of sintering time.

17. The dental zirconia-based green body of claim 15, further comprising the zirconia-based green body being simultaneously sinterable to form at least twenty units of dental restorations within one hour of sintering time.

18. The dental zirconia-based green body of claim 15, wherein the at least five units of dental restorations further comprise at least five of a single-unit crown, a multi-unit bridge, a full-arch All-on-X implant-supported prostheses, or combinations thereof.

19. The dental zirconia-based green body of claim 15, wherein:
the at least one other non-zirconium oxide equaling 7-20 weight percent (wt %) based on a total weight percent of the zirconia-based green body;
the zirconia-based green body being sinterable to form a sintered body with sintered grains; and
wherein all the sintered grains of the sintered body have a sintered grain size less than 1000 nano-meters (nm);
wherein a majority of the sintered grains of the sintered body have a sintered grain size greater than 100 nm.

20. A dental zirconia-based green body, comprising:
a zirconium oxide and at least one other non-zirconium oxide; and
the zirconia-based green body being sinterable to form at least one All-on-X dental restoration within two hours of sintering time and in ambient air without atmospheric control or hot isostatic pressure (HIP).

21. The dental zirconia-based green body of claim 20, further comprising:
the at least one other non-zirconium oxide equaling 8.8-20 weight percent (wt %) based on a total weight percent of the zirconia-based green body; and
the zirconia-based green body being sinterable to form a sintered body with an incisal or occlusal part corresponding to an incisal or occlusal area of a dental restoration;
wherein the incisal or occlusal part of the sintered body having a flexural strength greater than 800 mega-Pascals (MPa).
